(19) United States
(12) Patent Application Publication
(10) Pub. No.: US 2010/0049070 A1
(43) Pub. Date: Feb. 25, 2010

(54) DEVICE OF IMPROVING AUTOMATIC REAL-TIME HEART RATE VARIABILITY ANALYSIS USING ACCELEROMETER

(75) Inventors: TSAIR KAO, Taichung County (TW); CHING-WEI TSAI, Taipei County (TW)

Correspondence Address: WPAT, PC;INTELLECTUAL PROPERTY ATTORNEYS, 2030 MAIN STREET, SUITE 1300, IRVINE, CA, 92614, US

(73) Assignee: HUNGKUANG UNIVERSITY, Taichung County (TW)

(21) Appl. No.: 12/543,180

(22) Filed: Aug. 18, 2009

(30) Foreign Application Priority Data
Aug. 19, 2008 (TW) 097214862

(51) Int. Cl. A61B 5/0402

(52) U.S. Cl. 600/521

(57) ABSTRACT

The present invention provides a mobile ECG device by using accelerometer to improve real-time automatic analysis of heart rate variability, comprising: a data storage module; a microprocessor (MCU) module in which data are stored and accessed in the data storage module; an analog-to-digital converter module in which the data are transmitted to the microprocessor (MCU) module; an accelerometer peripheral hardware module in which the data are transmitted to the analog-to-digital converter module; and an electrocardiogram peripheral hardware module in which the data are transmitted to the analog-to-digital converter module.

### FIELD OF THE INVENTION

The present invention relates to a mobile ECG device that can improve real-time automatic analysis of heart rate variability (HRV).

### DESCRIPTION OF PRIOR ART

Heart rate plays a critical role in the human circulatory system, as its variation regulates and maintains the appropriate blood pressure and cardiac output. The time interval between each R-wave in the electrocardiogram (ECG) is the R-R interval, and the variation of R-R interval is the heart rate variability.

Short-term heart rate variability corresponds to the rapid control of heart rate mainly mediated by the autonomic nervous system. Frequency spectrum analysis of heart rate variability is an effective and quantifiable approach to differentiate between the heart rate control by the sympathetic nervous system from that by the parasympathetic (vagal) system.

In 1996, the standards, methods and clinical application for heart rate variability put forward by European Society of Cardiology and The North American Society of Pacing and Electrophysiology suggested that posture has a significant and direct impact on heart rate variability. For this reason, heart rate variability is often misdiagnosed because of different postures. The two societies established the physiological significance of HRV based on multiple clinical reports, LF ranges 0.04-0.15 Hz, HF ranges 0.15-0.4 Hz. The physiological meaning of various frequency as follows:

(1) High frequency (HF)—parasympathetic nervous system activity indicator;

(2) Low frequency (LF)—sympathetic and parasympathetic nervous system activity indicator;

(3) High-frequency percentage (HF %, equivalent to HF/(HF+LF))—parasympathetic nervous system activity indicator;

(4) Low-frequency percentage (LF %, equivalent to LF/(HF+LF))—sympathetic nervous system activity indicator;

(5) Low-frequency/high-frequency ratio (LF/HF)—sympathetic/parasympathetic balance indicator.

General devices record signals from the electrocardiogram and accelerometer, and may only record a few simple postures, ECG interpretation and data storage. These mobile systems do not perform automatic real-time computation of heart rate variability. The most salient feature of this new and improved device is that it factors in dynamic movements for automatic analysis of heart rate variability.

In the following literature, the systems in development are recording devices for ECG and accelerometer signals, with back-end analysis by human.

In 2000, J Ng et al., disclosed an electrocardiogram and an accelerometer storage device (J Ng et al. Sensing and documentation of body position during ambulatory ECG monitoring, Computers in Cardiology, 27:77-80, 2000). The system includes a two-axis accelerometer affixed on the chest, a single-axis accelerometer on the left thigh, electrocardiogram, digital-analog converters, and other related devices.

The prior art also disclosed an electrocardiogram and an accelerometer storage device (H L Chan et al., Segmentation of heart rate variability in different physical activities. Computers in Cardiology, 30:97-100, 2003; H L Chan et al., Heart rate variability characterization in daily physical activities using wavelet analysis and multilayer fuzzy activity clustering. IEEE Transactions on Biomedical Engineering, 53:133-139, 2006). The system also includes a two-axis accelerometer affixed on the chest, a single-axis accelerometer on the thigh, and data storage for the analysis of heart rate variability (non-real-time back-end analysis) to analyze the correlation between movements and heart rate variability.

In 2006, D M Karantonis et al., disclosed a device that uses a triaxial accelerometer affixed on the waist and measures user's movements through wireless transmission to the back-end system (D M Karantonis et al., Implementation of a real-time human movement classifier using a triaxial accelerometer for ambulatory monitoring. IEEE Transactions on Information Technology in Biomedicine, 10:156-166, 2006).

### BRIEF DESCRIPTION OF THE DRAWINGS

FIG. 1 is the operational flow chart of the modules in this invention.

FIG. 2 is the flow chart showing the computation in one embodiment of this invention.

FIG. 3 is the algorithm flow chart of the use of accelerometer and the computation of heart rate variability.

FIG. 4 is one embodiment of the algorithms used in this invention.

FIG. 5 is one embodiment of the wearing method for this invention.

FIG. 6 shows the computation results when wearing this invention in various postures.

### SUMMARY OF THE INVENTION

The present invention provides a mobile ECG device by using accelerometer to improve real-time automatic analysis of heart rate variability, comprising: a data storage module; a microprocessor (MCU) module in which data are stored and accessed in the data storage module; an analog-to-digital converter module in which the data are transmitted to the microprocessor (MCU) module; an accelerometer peripheral hardware module in which the data are transmitted to the analog-to-digital converter module; and an electrocardiogram peripheral hardware module in which the data are transmitted to the analog-to-digital converter module.

### DETAILED DESCRIPTION OF THE INVENTION

This invention relates to the use of accelerometer to dynamically analyze heart rate variability. The main feature of this new device is the utilization of microprocessor control unit (MCU) or micro-processor to perform automatic assessment of HRV, where the device could automatically and effectively identify the changes in heart rate variability in the same posture or movement. Continuous heart rate variability analysis could be of help in discovering abnormalities in the autonomic nervous system or other possible related anomalies. This invention presents a novel automatic HRV analysis device that performs automatic computation and considers dynamic movements.

At present, real-time heart rate variability computation and motion detection are two separated systems. Since heart rate variability combines the characteristic of many factors and is influenced by these various factors; hence, for automatic HRV computing system used in everyday life, it is difficult to identify the factors affecting the changes in heart rate variability to cause problems in HRV determination.

In the present new and improved device, user's movements and postures are effectively taken into consideration, assisting in determining changes in heart rate variability, especially in stationary states. In terms of daily use of the mobile system, long-term use can be more effective in producing improved determination of changes in HRV. This improved mobile ECG device includes automatic computation of heart rate variability and uses energy-efficient microprocessor control units to handle real-time analysis; the computation includes movement detection, time domain and frequency spectrum of heart rate variability computation and data storage capabilities. The improved new device must be equipped with a tri-axial accelerometer, where the movements or postures are obtained after the signals are processed by the microprocessor, effectively assisting in the determination of heart rate variability.

Conventional mobile ECG devices only provide long-term ECG records, and analysis the heart rate variation in the back-end computer. The present device includes not only the above-mentioned features, but also integrates real-time heart rate variability computation, adds accelerometer for movements determination, and takes into consideration on dynamic movements that have immense impact on the computation results. This device is a package that performs real-time automatic computation of heart rate variability with data storage capabilities. The method used in this invention could be applied to many microprocessors or digital signal processors.

The integration of accelerometer and heart rate variability allows real-time analysis of heart rate variability while the accelerometer could determine postures and movements, so that such the integration can assist changes in heart rate variability.

When in a stationary state, movements can be excluded from the factors of influence. When in motion, the accelerometers compute the activity scale for more effective assessment of heart rate variability. In the present invention, it found that, in general, during mild exercise, LF shows an increase while HF shows a decrease; during rigorous exercise, the obtained data have little significance, therefore are excluded. Consequently, using signals from the accelerometer to differentiate stationary state from the level of exercise intensity could help the determination of heart rate variability. Clinically speaking, accelerometers could be jointly used with electrocardiogram monitoring to detect occasional arrhythmia.

This invention relates to a mobile ECG device that uses accelerometer to improve real-time automatic heart rate variability analysis, comprising (1) a data storage module. Others comprise a power supply module that could be connected to external power supply or by using batteries or a combination of the two; (2) a microprocessor (MCU) module where the data can be stored and accessed in the data storage module; (3) an analog-to-digital converter module for the purpose of conversion of analog signals to digital signals for the operation of micro-processor (MCU) module where the data can be transmitted to the microprocessor (MCU) module; (4) an accelerometer peripheral hardware module, e.g. a tri-axial accelerometer where the data can be transmitted to the analog-to-digital converter (ADC) module; and (5) an electrocardiogram(ECG) peripheral hardware module, e.g. electrocardiogram and electrodes where the data can be transmitted to the analog-to-digital converter module.

The processor (MCU) module in this invention comprises: (1) R-wave detection computation where the R-wave interval of heart beat cycles is measured and the data can be used for computation of HRV parameters; (2) computation of HRV parameters, where heart beat interval is obtained using ECG or pulse measurement to obtain the continuous changes in heart rate, and the data could be used for the computation of integrated determination; (3) movement determination computation uses data from the accelerometer to process and determine the current posture and level of exercise intensity for integrated determination computation; (4) the integrated determination computation utilizes personal movements as parameters to calibrate the errors in heart rate variability caused by different postures and the HF and LF of various level of exercise intensity, and the data can be used for storage peripheral control; and (5) data storage peripheral control uses control chips, where the data can be stored and accessed in the data storage module, such as flash memory.

The above shows that this invention of a mobile ECG device is absolutely feasible and possesses new and improved features. In order to allow those skilled in the art to understand the content of the invention and the embodiment, and to easily comprehend the related purpose and advantages in the content, claims and drawings disclosed in this application, the embodiment will detail the features and advantages of this invention. Please refer to the drawings and descriptions for the content of this invention and embodiment examples. In fact, this invention may be embodied in a variety of forms and should not be inferred to be limited by the examples given in the texts.

### Example

### Example 1

### Operational Procedure

First, this invention was placed in a waist bag, while the electrodes were affixed to the lower chest area. After making sure the invention was turned on, follow the instructions and used accelerometer peripheral hardware module 10 to obtain movement data and data from ECG peripheral hardware module 20. The data were converted by analog-to-digital converter module 30, and processed by microprocessor (MCU) module 40, containing data of R-wave detection computation 41, movement determination computation 42 and computation of HRV parameters 43. Then, integrated determination computation 44 was carried out. Finally, through data storage peripheral control 45, the data were stored in the data storage module 50 for the purpose of follow-ups.

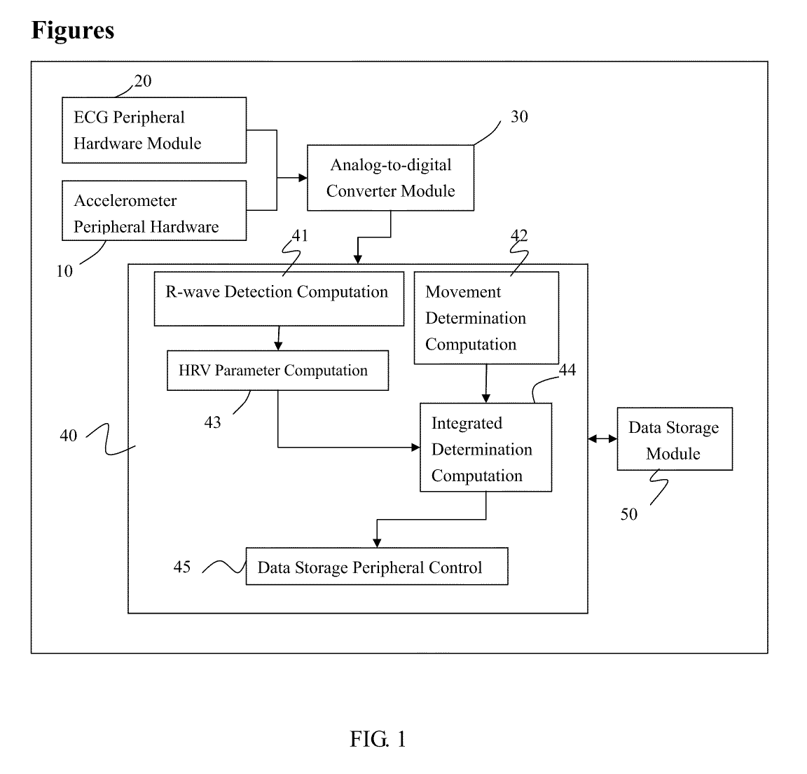

FIG. 1 represented the operational flow chart of this invention. In detail, the operational procedure of this invention comprised: (1) ECG signal input for automatic detection of R-wave; (2) real-time computation of heart rate variability of time domain and frequency spectrums using parameter algorithms; (3) using accelerometer signals to determine movements and postures; (4) using flash memory as data storage device for read and write; and (5) method for determining heart rate variability with assistance of movement detection.

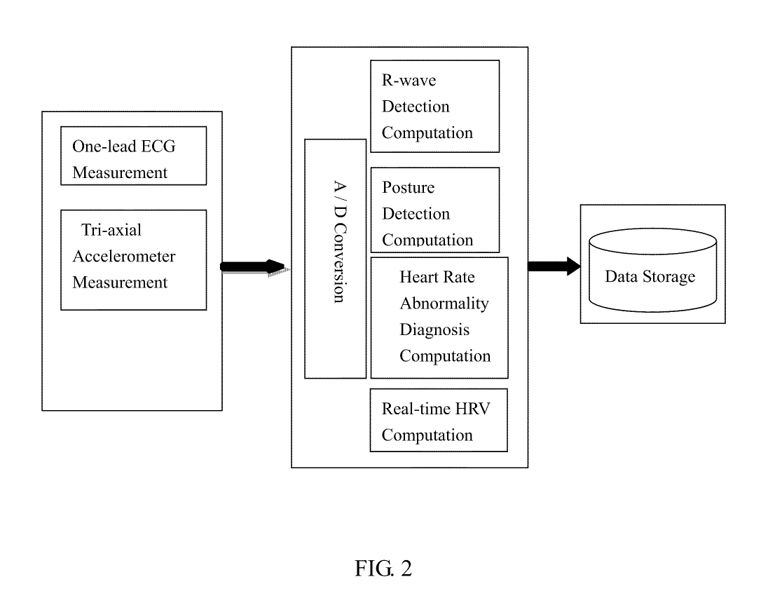

FIG. 2 represented the flow chart showing the computation in one embodiment of this invention. The left-hand portion was the hardware, requiring at least accelerometer peripheral hardware module 10 (accelerometer) and ECG peripheral hardware module 20 (one lead electrocardiogram). The middle portion was the microprocessor control unit module 40, where the microprocessor (MCU) module 40 comprised an analog-to-digital converter module 30 incorporating at least the algorithms for R-wave detection computation, movement determination, data storage and real-time HRV computation. The storage device was data storage module 50 (flash memory or other read-and-write non-volatile memory).

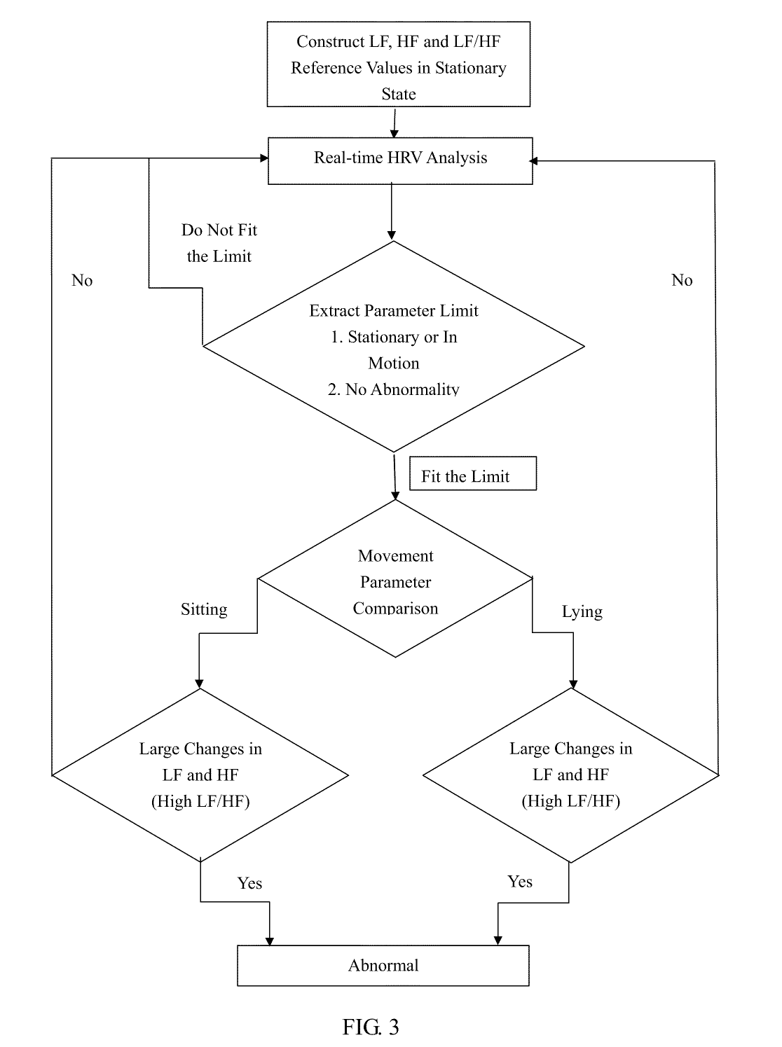

FIG. 3 was the algorithm flow chart for heart rate variability by the use of accelerometer. First, a personal reference value for each posture was constructed, and then through the calibration by automatic HRV computation for movements and postures, the obtained LF/HF ratio was used to determine whether abnormalities are present.

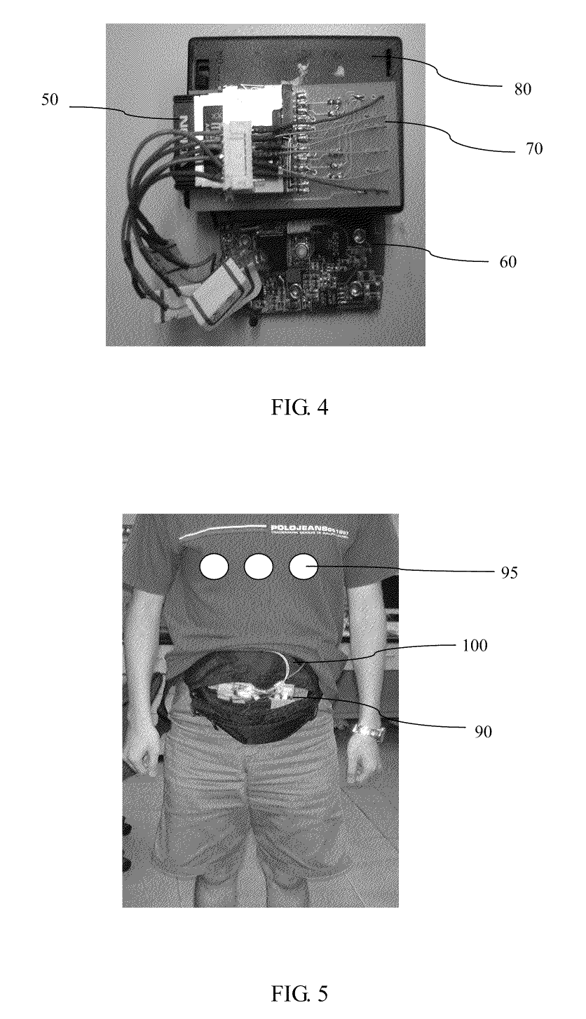

FIG. 4 was one embodiment of this invention, where circuit board 60 made with accelerometer peripheral hardware module 10 (accelerometer), ECG peripheral hardware module 20 and microprocessor (MCU) module 40 was used to accept the data from the electrodes. Coupled with accelerometer for integrated computation, the data were sent to circuit board 70 of data storage peripheral control 45 and stored in the data storage module 50. The power came from power supply box 80 installed with (rechargeable) batteries.

FIG. 5 was one embodiment of this invention. For use of this invention 90, three electrodes 95 were affixed to the lower chest area, as in general use of electrodes for electrocardiogram. This invention was placed in the waist bag 100 for calculation of heart rate variability in various postures.

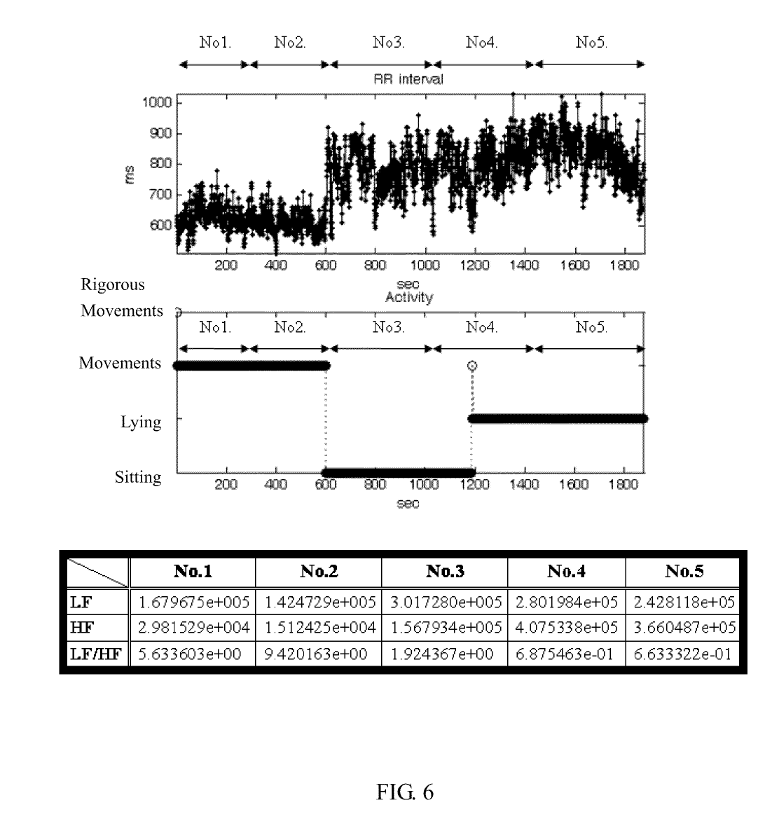

FIG. 6 was the calculation results of each posture when wearing this invention. Using R-wave algorithms to identify R-R interval, together with the accelerometer signal for movement determination, the movements of heart rate variability of time and frequency spectrums computed for each interval were obtained. The content of the table corresponded to the value from frequency spectrum analysis obtained in real-time for each interval computed by automatic heart rate (the display value was the raw value from SD storage card calculated to the sixth decimal point).

In short, this invention was used for constructing personal basic movement parameters. Therefore, when wearing the invention, it performed real-time detection of changes in heart rate while taking personal movement parameter into account for adjustments. The data were stored in the memory for follow-up use.

While the invention had been disclosed and illustrated with reference to a preferred embodiment thereof, it would be understood that various changes in the details, materials and arrangements of the parts which had been described and illustrated in order to explain the nature of this invention might be made by those skilled in the art without departing from the principle and scope of the invention as expressed in the following claims.

### Description of Major Component Notations

10 accelerometer peripheral hardware module
20 ECG peripheral hardware module
30 analog-to-digital converter (ADC) module
40 microprocessor (MCU) module
41 R-wave detection computation
42 movement determination computation
43 computation of HRV parameters
44 integrated determination computation
45 data storage peripheral control
50 data storage module
60 circuit board—accelerometer peripheral hardware module, ECG peripheral hardware module and microprocessor (MCU) module
70 data storage peripheral control circuit board
80 power supply box
90 embodiment of this invention
95 electrodes
100 waist bag

What is claimed is:

1. A mobile ECG device by using accelerometer to improve real-time automatic analysis of heart rate variability, comprising:
a data storage module;
a microprocessor (MCU) module in which data are stored and accessed in the data storage module;
an analog-to-digital converter module in which the data are transmitted to the microprocessor (MCU) module;
an accelerometer peripheral hardware module in which the data are transmitted to the analog-to-digital converter module; and
an electrocardiogram peripheral hardware module in which the data are transmitted to the analog-to-digital converter module.

2. The device of claim 1, further comprising a power supply module.

3. The device of claim 2, wherein the power supply module is connected to an external power source or a battery or a combination of the power source and battery.

4. The device of claim 1, wherein the accelerometer peripheral hardware module further comprises a tri-axial accelerometer.

5. The device of claim 1, wherein the ECG peripheral hardware module further comprises an electrocardiogram and electrodes.

6. The device of claim 1, wherein the analog-to-digital converter module converses analog signal to digital signal and is used for operations in the microprocessor (MCU) module.

7. The device of claim 1, wherein the microprocessor (MCU) module further comprises:
R-wave detection computation where the data are used for computation of heart rate variability parameters;
computation of heart rate variability parameter where the data are used for integrating determination computation;
movement determination computation where the data are used for integrating determination computation;
integrated determination computation where the data are used for data storage peripheral control; and
data storage peripheral control where the data are stored and accessed in the data storage module.

8. The device of claim 7, wherein the R-wave detection computation is the R-wave interval of the heart beat cycle.

9. The device of claim 7, wherein the parameter computation of heart rate variability is the heart beat interval obtained using ECG or pulse measurement to obtain time domain and frequency spectrum parameters of continuous heart beat in velocity change.

10. The device of claim 7, wherein the movement determination computation uses the data from the accelerometer to process the posture and level of exercise intensity of the current state.

11. The device of claim 7, wherein the integrated determination computation utilizes personal movements as parameters to calibrate the errors in heart rate variability caused by different postures and the HF and LF of various level of exercise intensity.

12. The device of claim 7, wherein the data storage peripheral control is a control chip.

13. The device of claim 1, wherein the data storage module is a flash memory.
